(19) United States
(12) Patent Application Publication
(10) Pub. No.: US 2006/0195102 A1
(43) Pub. Date: Aug. 31, 2006

(54) Apparatus and method for treatment of spinal conditions

(76) Inventor: Hugues F. Malandain, Mountain View, CA (US)

Correspondence Address: COOLEY GODWARD LLP;ATTN: PATENT GROUP, THE BOWEN BUILDING, 875 15TH STREET, N.W. SUITE 800, WASHINGTON, DC, 20005-2221, US

(21) Appl. No.: 11/059,526

(22) Filed: Feb. 17, 2005

(51) Int. Cl. A61F 2/44; A61B 17/58; A61F 2/30

(52) U.S. Cl. 606/72; 623/17.16

(57) ABSTRACT

An apparatus includes a first clamp having a first end and a second end. The second end of the first clamp is configured to engage a first spinous process. A second clamp has a first end and a second end. The second end of the second clamp is configured to engage a second spinous process spaced apart from the first spinous process. A connector is coupled to the first end of the first clamp and the first end of the second clamp.

### BACKGROUND

The invention relates generally to the treatment of spinal conditions. More specifically, the invention relates to an apparatus and method for the treatment of spinal compression.

A significant back condition that impacts many individuals is spinal stenosis. Spinal stenosis is a narrowing of the spinal canal that causes compression of the spinal cord. Spinal stenosis can cause pain, weakness, numbness, burning sensations, tingling, and in particularly severe cases, may cause loss of bladder or bowel function or paralysis. Spinal stenosis is a progressive narrowing of the spinal canal. Each vertebra in the spinal column has an opening that extends through it. The openings are aligned vertically to form the spinal canal. The spinal cord runs through the spinal canal. As the spinal canal narrows, the spinal cord and nerve roots extending from the spinal cord and between adjacent vertebrae are compressed and may become inflamed. Pressure on the spinal cord and nerve roots causes pain. The legs, calves and buttocks are most commonly affected by spinal stenosis, however, the shoulders and arms may also be affected.

Mild cases of spinal stenosis may be treated with rest or restricted activity, non-steroidal anti-inflammatory drugs (e.g., aspirin), corticosteroid injections (epidural steroids), and physical therapy. Some patients find that bending forward, sitting or lying down may help relieve the pain. This may be due to bending forward creating more vertebral space which may temporarily relieve nerve compression. Since spinal stenosis is a progressive disease, the source of pressure may have to be surgically corrected (decompressive laminectomy) as the patient gradually worsens and has increasing pain. The surgical procedure can remove bone and other tissues that have impinged upon the spinal canal or put pressure on the spinal cord. Two adjacent vertebrae may also be fused during the surgical procedure to prevent an area of instability, improper alignment or slippage, such as that caused by spondylolisthesis. Surgical decompression can relieve pressure on the spinal cord or spinal nerve by widening the spinal canal to create more space. This procedure requires that the patient be given a general anesthesia as an incision is made in the patient to access the spine to remove the areas that are contributing to the pressure. This procedure, however, may result in blood loss, an increased chance of significant complications, and usually results in an extended hospital stay.

Minimally invasive procedures have been developed to provide access to the space between adjacent spinous processes such that major surgery is not required. Such known procedures, however, may not be suitable in conditions where the spinous processes are severely compressed.

Thus, a need exists for improvements in the treatment of spinal conditions such as spinal stenosis.

### SUMMARY OF THE INVENTION

An apparatus includes a first clamp having a first end and a second end. The second end of the first clamp is configured to engage a first spinous process. A second clamp has a first end and a second end. The second end of the second clamp is configured to engage a second spinous process spaced apart from the first spinous process. A connector is coupled to the first end of the first clamp and the first end of the second clamp.

### BRIEF DESCRIPTION OF THE DRAWINGS

FIG. 1 is a schematic illustration of an apparatus according to an embodiment of the invention.

FIG. 2 is a front plan view of an apparatus according to an embodiment of the invention and a portion of a spine.

FIG. 3 is a cross-sectional view of a component of the apparatus and the portion of the spine illustrated in FIG. 2, taken along line 3-3 in FIG. 2.

FIG. 4 is a side plan view of the apparatus illustrated in FIG. 2.

FIG. 5 is a side plan view of a component of the apparatus illustrated in FIG. 2.

FIG. 6 is a front plan view of the component of the apparatus illustrated in FIG. 5.

FIG. 7 is a partial cross-sectional view of a detachable trocar tip for use with an apparatus according to an embodiment of the invention in a first configuration.

FIG. 8 is a partial cross-sectional view of the detachable trocar tip for use with the apparatus according to an embodiment of the invention in a second configuration.

FIG. 9 is a partial exploded view of a detachable trocar tip for use with the apparatus according to an embodiment of the invention.

FIG. 10 is a side plan view of an apparatus according to another embodiment of the invention.

FIG. 11a is a perspective view of an apparatus according to an embodiment of the invention.

FIG. 11b is an exploded view of a portion of the apparatus illustrated in FIG. 11a.

FIG. 11c is an exploded view of a portion of the apparatus illustrated in FIG. 11a.

FIG. 12 is a perspective view of a spacer configured to be inserted between adjacent spinous processes according to an embodiment of the invention.

FIG. 13 is a side view of a spacer according to an embodiment of the invention in a first configuration inserted between adjacent spinous processes.

FIG. 14 is a side view of the spacer illustrated in FIG. 12 in a second configuration inserted between adjacent spinous processes.

FIGS. 15-17 are illustrations of spacers according to alternative embodiments of the invention.

FIG. 18 is a side view of a spacer according to an alternative embodiment of the invention in a first configuration.

FIG. 19 is a side view of the spacer illustrated in FIG. 18 in a second configuration inserted between adjacent spinous processes.

FIG. 20 is a side view of a spacer according to a further alternative embodiment of the invention inserted between adjacent spinous processes.

FIG. 21 is a side view of a spacer according to another alternative embodiment of the invention inserted between adjacent spinous processes.

### DETAILED DESCRIPTION

An apparatus includes a first clamp having a first end and a second end. The second end of the first clamp is configured to engage a first spinous process. A second clamp has a first end and a second end. The second end of the second clamp is configured to engage a second spinous process spaced apart from the first spinous process. A connector is coupled to the first end of the first clamp and the first end of the second clamp.

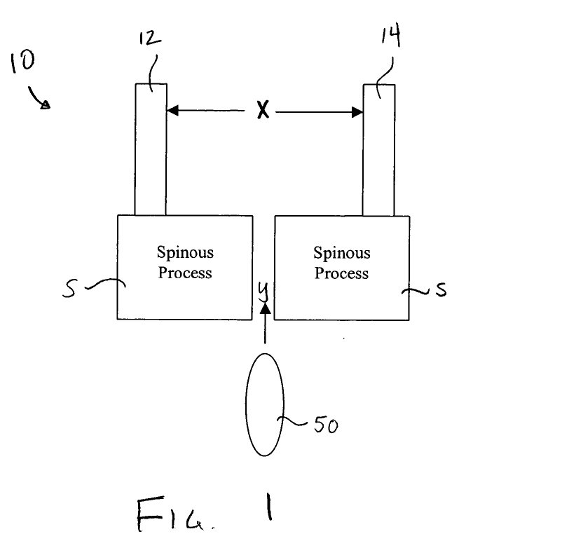

FIG. 1 is a schematic illustration of a medical device according to an embodiment of the invention attached to two adjacent spinous processes. The apparatus 10 includes a first clamp 12 configured to be coupled to a first spinous process S and a second clamp 14 configured to be coupled to a second spinous process S. The first clamp 12 and the second clamp 14 are configured to be moved apart from one another in the direction indicated by arrows X. As the first clamp 12 and the second clamp 14 are moved apart, an opening between adjacent spinous processes S expands. An insert 50 can be inserted between the spinous processes S in the direction indicated by arrow Y to maintain the opening between the spinous processes S. The clamps 12, 14 engage the spinous processes S with sufficient force such that when the clamps 12, 14 are spread apart, they cause lateral displacement of the spinous processes S.

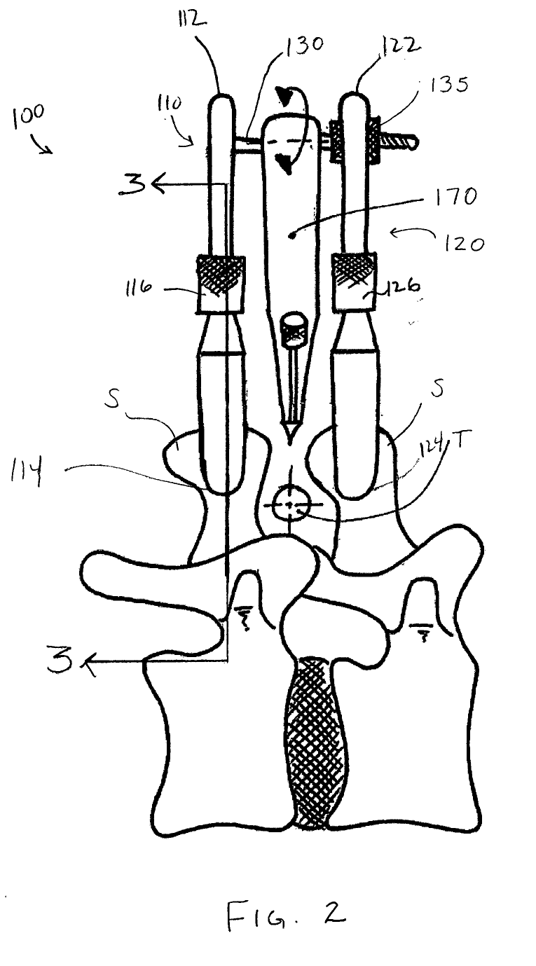

FIG. 2 is a side view of a medical device according to an embodiment of the invention coupled to a portion of a spine. The tissue surrounding the spine is not illustrated for the sake of clarity. The medical device 100 includes a first clamp 110 and a second clamp 120. The first clamp 110 has a proximal end 112 and a distal end 114. The distal end 114 of the first clamp 110 is configured to engage a first spinous process S. The second clamp 120 has a first end 122 and a second end 124. The second end 124 of the second clamp 120 is configured to engage a second spinous process S that is spaced apart from the first spinous process S.

A connector 130 is coupled to the proximal end 112 of the first clamp 110 and the first end 122 of the second clamp 120. The position of the connector 130 relative to the first clamp 110 and the second clamp 120 can be adjusted such that the distance between the first clamp 110 and the second clamp 120 can be adjusted. In other words, the connector 130 is reconfigurable between a first configuration and a second configuration. The first clamp 110 is a first distance from the second clamp 120 when the connector 130 is in its first configuration and is a second distance from the second clamp 120 when the connector 130 is in its second configuration.

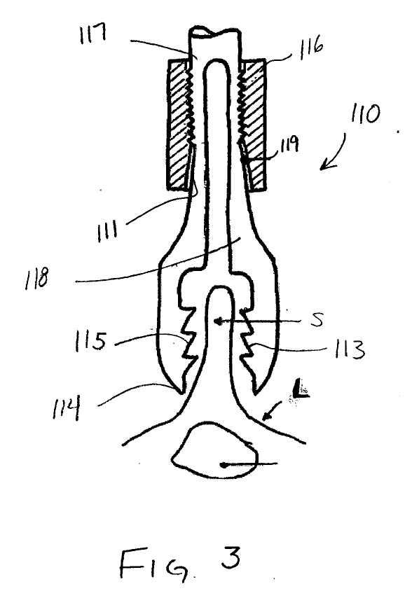

Referring to FIG. 3, in which the first clamp 110 is illustrated, the first clamp 110 includes a first jaw 115 and a second jaw 113 opposite the first jaw 115. The first jaw 115 and the second jaw 113 are configured to be movable between a first configuration and a second configuration. The first jaw 115 and the second jaw 113 are closer together in the second configuration than in the first configuration. In the second configuration, the first jaw 115 and the second jaw 113 engage the spinous process S with sufficient force to substantially maintain the orientation of the first clamp 110 and the second clamp 120 with respect to the spinous process S when the connector 130 is moved to its second configuration, thereby spreading the spinous processes S. The second clamp 120 has a similar configuration, but is not illustrated for ease of reference. The material of the jaws 115, 113 are such that they can sufficiently engage the spinous processes S as described, but to not damage the spinous processes. Adequate materials include, for example, stainless steel, polyetheretherketone (PEEK), carbon fiber, ultra-high molecular weight (UHMW) polyethylene, etc. The material can have a tensile strength similar to or higher than that of bone. In some embodiments, the clamp 120 can be manufactured from stainless steel and a coating and/or an over-mold or over-layer of PEEK or carbon fiber can be applied to the jaws 115, 113.

In some embodiments, the medical device 100 is used to spread adjacent spinous processes of severely compressed vertebrae. Additionally, the medical device 100 stabilizes the spinous processes during procedures without penetrating the vertebrae.

In some embodiments, the first clamp 110 includes a first arm 117 and a second arm 118 and a tension member 116. The first arm 117 and second arm 118 can be resiliently coupled such that as tension member 116 is advanced towards the distal end 114 of the clamp 110, the first arm 117 and the second arm 118 are moved towards one another, but as the tension member 116 is moved away from the distal end 114 of the clamp 110, the first arm 117 and the second arm 118 return to their default position (i.e., spaced apart).

The tension member 116 is configured to move the first jaw 115 and the second jaw 113 between their first configuration and their second configuration as the first arm 117 and the second arm 118 move towards one another. As the tension member 116 is moved towards the first jaw 115 and the second jaw 113, the first jaw 115 and the second jaw 113 engage the spinous process S. In some applications, a distal end 114 of the clamp 110 is positioned adjacent the lamina L of the vertebra to which it is coupled. In some embodiments, the clamp 110 is attached close to the lamina L to minimize the lever arm on the spinous process. The distal end 114 of clamp 110 need not penetrate the lamina L.

In an alternative embodiment, the tension member includes threads that engage threads on the first clamp. In such an embodiment, the tension member is moved along the length of the first clamp by turning the tension member. Returning to FIG. 3, the tension member 116 may optionally include a tapered portion 119 that matingly engages a tapered portion 111 of first clamp 110. Such a configuration can ensure appropriate distribution of the forces to the spinous process S. The second clamp 120 is similarly configured and includes a tension member 126 and opposing jaws.

A swing arm 170 is pivotably coupled to the connector 130 between the first clamp 110 and the second clamp 120. The swing arm 170 has an arcuate portion 173 and travels along a range of motion. The arcuate portion 173 of the swing arm 170 has a first end 175 and a second end 177.

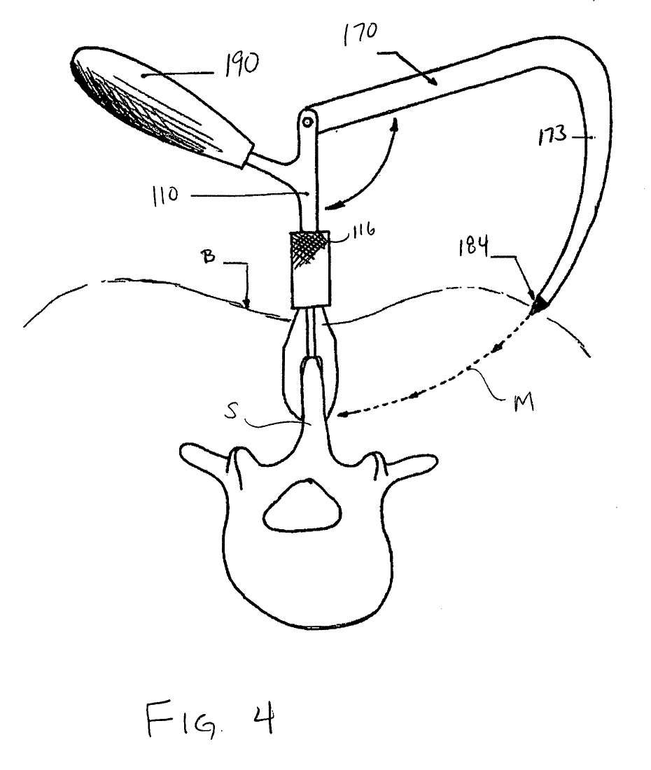

As best seen in FIGS. 4 and 5, the second end 177 of the arcuate portion 173 of swing arm 170 is configured to receive a working tool 184, such as, for example, a pointed trocar tip. The swing arm 170 defines an opening 174 in which at least a portion of the working tool 184 is received. In some embodiments, the opening 174 extends along the entire length of the arcuate portion 173 between the first end 175 and the second end 177. In some embodiments, an optional handle 190 can be coupled to the first clamp 110 and/or the second clamp 120 to facilitate insertion of the clamps 110, 120 and increase stability of the apparatus 100 during use.

The working tool 184 is coupled to a guide wire 186. The guide wire 186 has a first end 181 and a second end 183. The second end 183 of the guide wire 186 is coupled to the working tool 184. A retainer 182 (discussed in detail below) is coupled to the first end 181 of the guide wire 186 and is configured to maintain the position of the working tool 184 with respect to the swing arm 170. The retainer 182 is matingly received in a recess 172 in the swing arm 170. The guide wire 186 is received in the opening 174 defined in the swing arm 170. The guide wire is received in the opening 174 through a channel 176 defined in the swing arm 170 as best seen in FIG. 6. In some alternative embodiments, the guide wire does not extend through the opening 174 of the swing arm 170. In yet other alternative embodiments, the guide wire is not present.

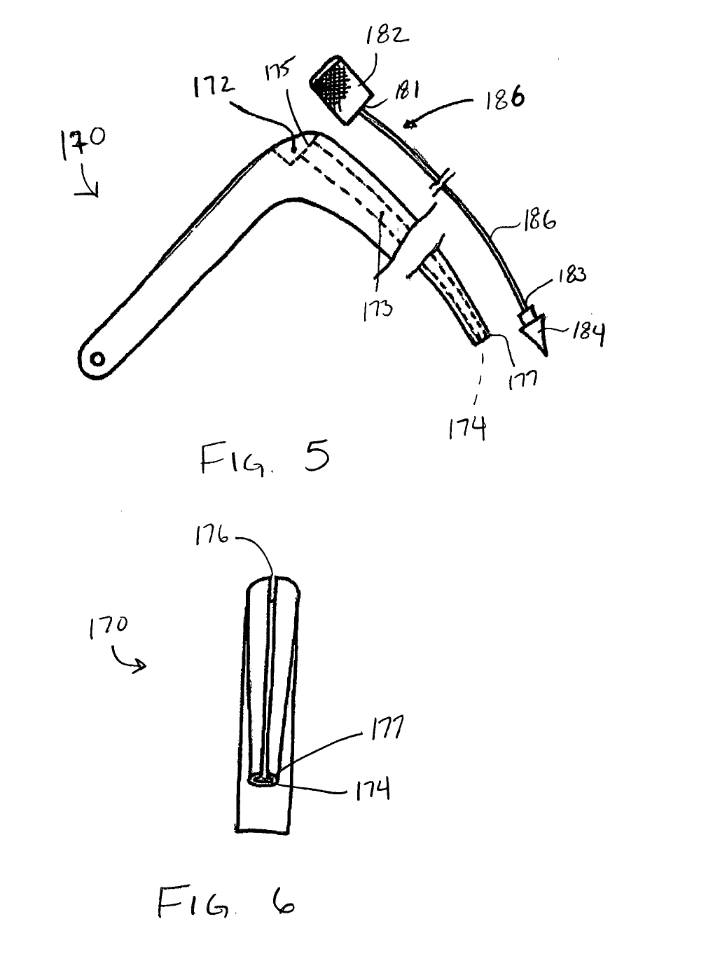

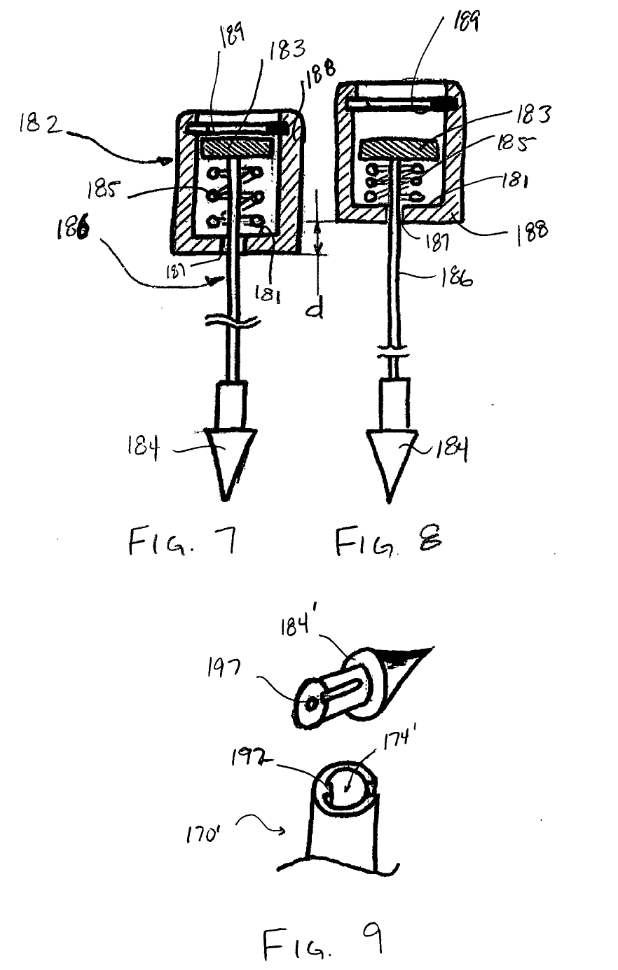

FIGS. 7 and 8 illustrate the retainer 182 in a first configuration and a second configuration, respectively. The retainer 182 includes a housing 188 that defines an opening 187 through which guide wire 186 is movably disposed. The guide wire 186 is coupled to a retention member 183. The retention member 183 is biased towards a first end 189 of housing 188 by a spring 185. The spring 185 is between a second end 181 of the housing 188 and the retention member 183.

In use, when the retainer 185 is in the first configuration (FIG. 7), the working tool is maintained in the swing arm 170. When the retainer 182 is moved to its second configuration (FIG. 8), the working tool 184 can be removed from the swing arm 170. When moved to the second configuration, the retainer 182 is displaced a distance d, thereby increasing the effective length of the guide wire 186, allowing movement of the working tool 184 with respect to the end of the swing arm 170. In some embodiments, the distance d is approximately the same as the length of the portion of the working tool 184 received in the swing arm 170.

As shown in FIG. 9, a working tool 184′ is inserted into an opening 174′ defined by a swing arm 170′. The swing arm 170′ includes a projection 192 within opening 174′ that mates with a recess 197 on working tool 184′.

Returning to FIGS. 2-5, in use, a first clamp 110 is inserted through a body B and coupled to a spinous process S. The tension member 116 is moved towards the distal end 114 of the first clamp to engage the first jaw 115 and the second jaw 113 with the spinous process S. The second clamp 120 is then inserted and similarly coupled to the adjacent spinous process S. The connector 130 is actuated to increase the distance between the first clamp 110 and the second clamp 120, thereby separating the adjacent spinous processes S. Once the spinous processes S are separated, the swing arm 170 is moved through its range of motion M.

The swing arm 170 is moved from a location outside a body B through a range of motion M (see, e.g., FIG. 4). The swing arm 170 enters the body B and moves through range of motion M until it is at target T (see, e.g., FIG. 2) between adjacent spinous processes S.

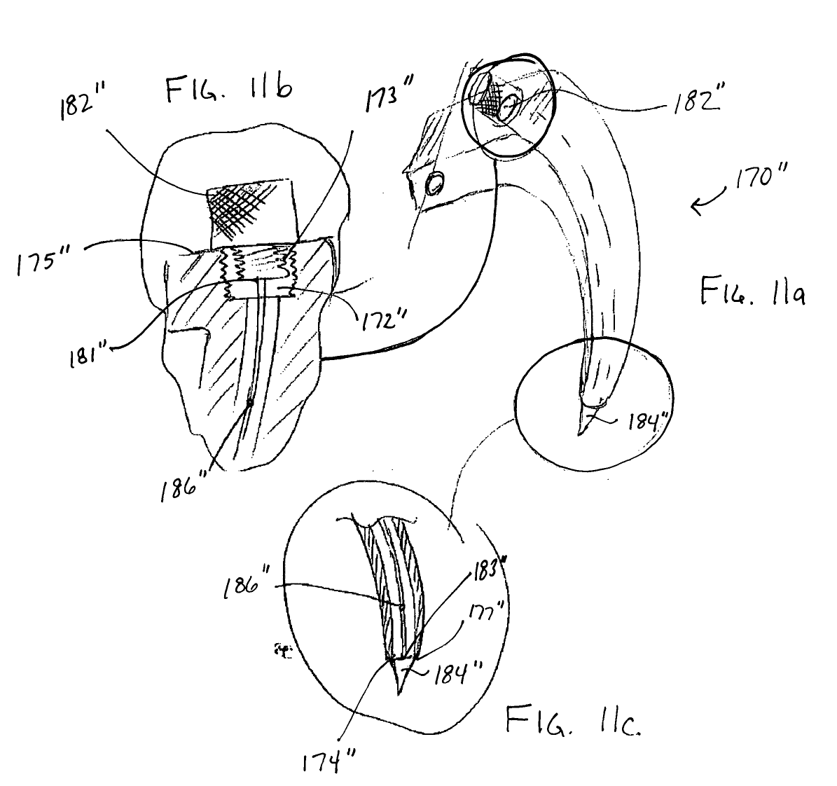

The movement of the swing arm 170 into the body defines a path within the tissue (not illustrated). The tissue is penetrated by a pointed projection (i.e., working tool 184). The path M defined by the swing arm 170 includes the target T between the adjacent spinous processes S. Once the path is defined, the swing arm 170 can be removed and a spacer 500 (see FIG. 12), discussed in detail below, can be inserted between the adjacent spinous processes S. In some embodiments of the invention, the spacer 500 can be removably attached to the swing arm 170, inserted into the body and then removed from the swing arm 170.

A medical device 200 according to an embodiment of the invention is illustrated in FIG. 10. Medical device 200 includes a handle 290 coupled to an arm 270. The arm 270 has a first end 275 and a second end 277 and defines an opening 274 along its length. A working tool 284 can be received within opening 274 adjacent the second end 277. The arm 270 also includes a recess 272 to receive a retainer (not illustrated) similar to retainer 185 discussed above. Medical device 200 is inserted between adjacent spinous process in a manner similar to swing arm 170 discussed above. The depth and placement of the arm 270, however is determined by the user of the medical device 200. Such a medical device can be used with or without the benefit of the clamps 110, 120 discussed above. In other words, the medical device 200 can be inserted between adjacent spinous processes S without first separating the spinous processes S.

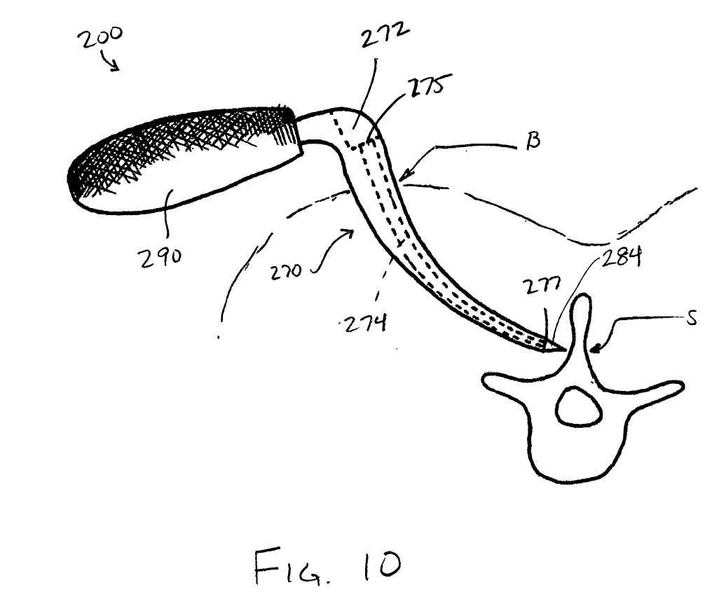

An alternative swing arm 170″ for use with medical device 100 according to an embodiment of the invention is illustrated in FIGS. 111a-111c. As best seen in FIGS. 11a and 11c, the second end 177″ of swing arm 170″ is configured to receive a working tool 184″, such as, for example, a pointed trocar tip. The swing arm 170″ defines an opening 174″ in which at least a portion of the working tool 184″ is received. In some embodiments, the opening 174″ extends along the entire length of the swing arm 170″ between the first end 175″ and the second end 177″ to define a passageway or lumen. The opening 174″ is slightly larger than the diameter of the working tool 184″ such that the working tool 184″ is positioned within the opening 174″ during use.

The working tool 184″ is coupled to a wire 186″. The wire 186″ has a first end 181″ and a second end 183″. The second end 183″ of the wire 186″ is coupled to the working tool 184″. A retainer 182″ (discussed in detail below) is coupled to the first end 181 ″ of the wire 186″ and is configured to maintain the position of the working tool 184″ with respect to the swing arm 170″. In some embodiments, the wire 186″ is substantially rigid such that the working tool 184″ is not retracted into the opening 174″ when force is imparted against the working tool 184″.

The retainer 182″ is received in a recess 172″ in the swing arm 170″. The retainer 182″ is maintained in the recess 172″ using threaded fasteners 173″. In some alternative embodiments, the wire 186″ does not extend through the opening 174″ of the swing arm 170″. In yet other alternative embodiments, the wire 186″ is not present.

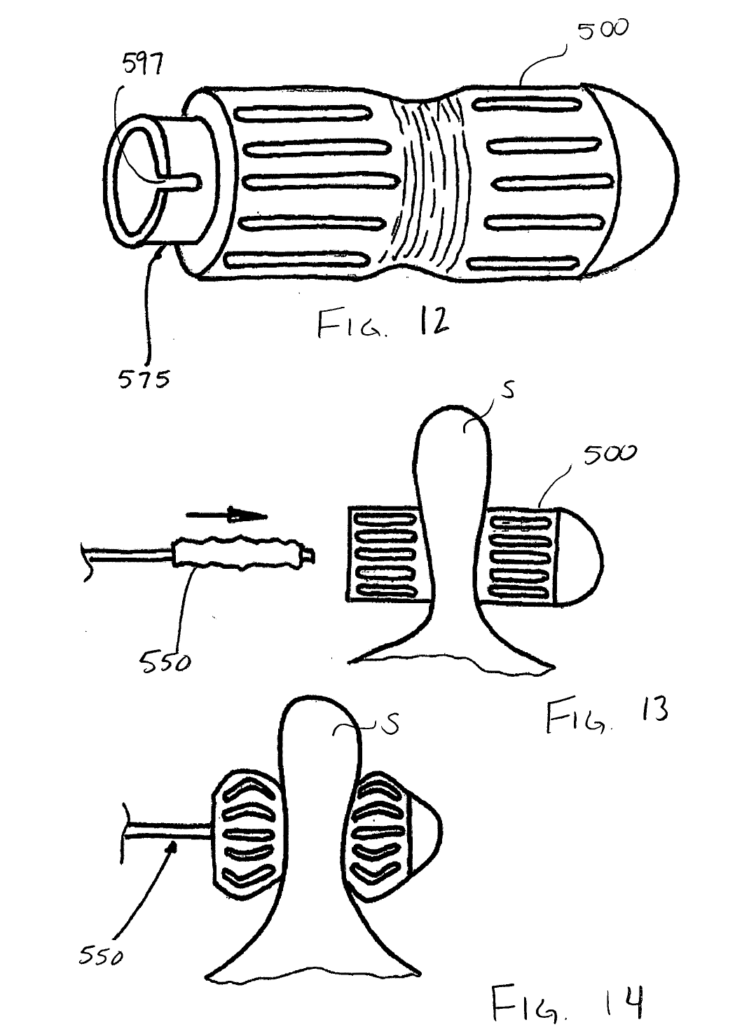

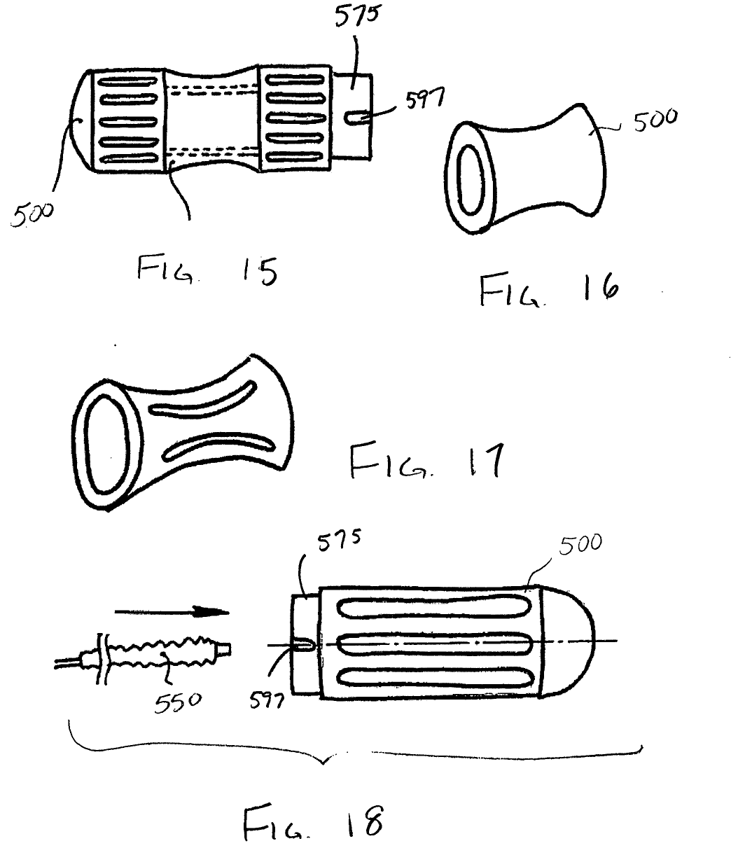

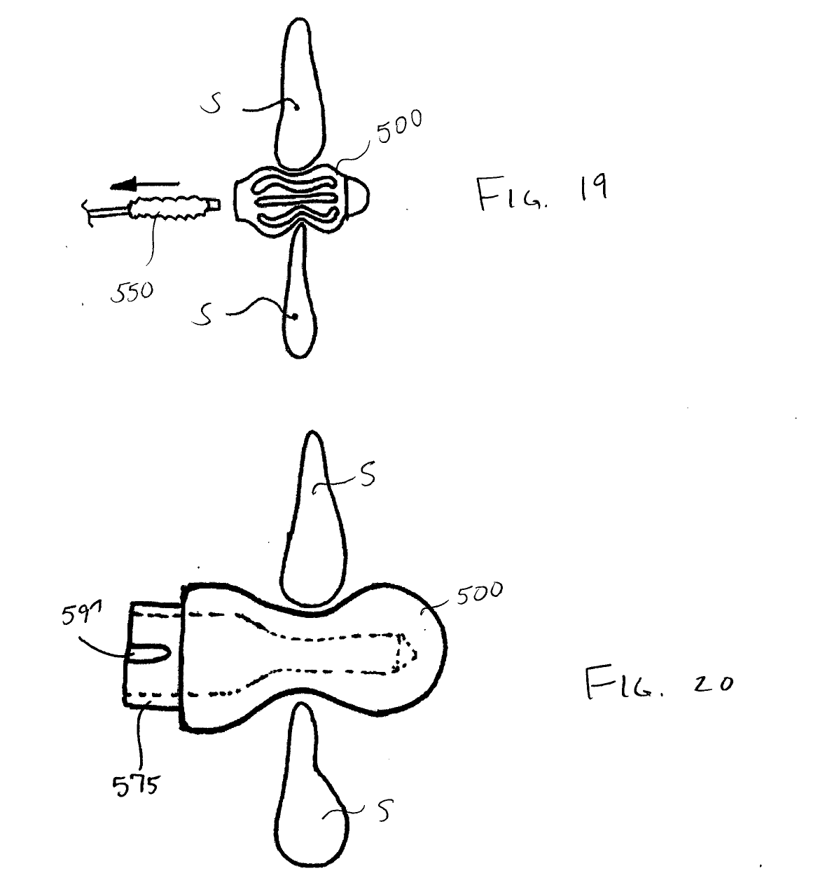

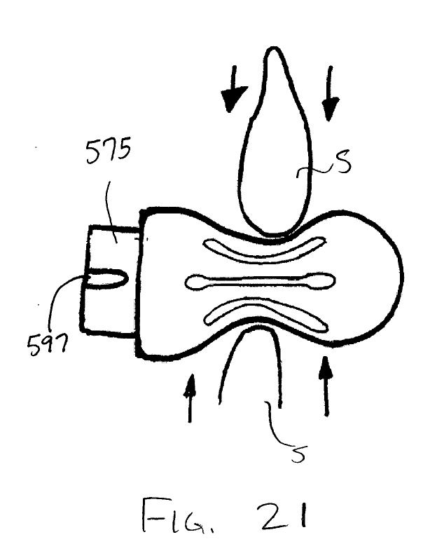

FIGS. 12-21 illustrate various spacers 500 that can be inserted between adjacent spinous processes S. Once the spacer 500 is inserted between the spinous processes S, depending upon the type of spacer 500, the spacer 500 can be deformed to be held in place. For example, in some embodiments, a balloon actuator 550 can be inserted into the spacer and expanded, thereby expanding the ends of the spacer 500 to retain the spacer 500 between the spinous processes S (see, e.g., FIGS. 13, 14 and 18). Once the spacer 500 is expanded, the balloon actuator 550 can be deflated and removed (see, e.g., FIG. 19).

In some embodiments of the invention, the spacer 500 includes an end portion 575 that includes a recess 597 that is configured to mate with the projection 192 on swing arm 170′ (see FIG. 9).

### CONCLUSION

While various embodiments of the invention have been described above, it should be understood that they have been presented by way of example only, and not limitation. Thus, the breadth and scope of the invention should not be limited by any of the above-described embodiments, but should be defined only in accordance with the following claims and their equivalents.

The previous description of the embodiments is provided to enable any person skilled in the art to make or use the invention. While the invention has been particularly shown and described with reference to embodiments thereof, it will be understood by those skilled in art that various changes in form and details may be made therein without departing from the spirit and scope of the invention.

For example, although the swing arm 170 is described as having an arcuate portion, in alternative embodiments of the invention, the entire swing arm 170 may have an arcuate configuration. Additionally, the opening defined in the swing arm 170 may extend the entire length of the swing arm 170.

Although the swing arm 170 is described and illustrated as having a circular opening at its end, in alternative embodiments, the opening can be any shape and the shape of the portion of the working tool and/or spacer can be shaped to be matingly engage the opening of the swing arm.

Although the connection between the swing arm and the working tool are shown with the swing arm being the female component and the working tool being the male component, in alternative embodiments, the orientation of the male/female relationship may be reversed.

Although the first arm 117 and second arm 118 of the first clamp 110 are described as being resiliently coupled, in alternative embodiments of the invention, the first arm 117 and the second arm 118 are pivotably or hingedly coupled.

Although the first clamp and second clamp are disclosed as having jaws that engage opposite sides of a spinous process, in alternative embodiments, the first clamp and second clamp may include other configurations to engage the spinous process such as, for example, suction, adhesive, pins/projections, etc.

While the first clamp and second clamp are disclosed as being movable with respect to one another, in alternative embodiments, the first clamp or the second clamp may be fixed in position, with the other clamp moving relative to the fixed clamp.

While the first arm and the second arm of the clamp are shown as being resiliently biased apart from one another, in alternative embodiments, the first arm and the second arm can be manually moved towards and away from one another using a different configuration (e.g., scissor configuration).

Although embodiments are disclosed that illustrate the wire being coupled to the swing arm using a retainer, in alternative embodiments, a retainer need not be used. The wire can be coupled to the swing arm using other retention methods, such as, for example, a slit in which the wire can be clamped.

Additionally, although the working tool 184 is disclosed as a trocar tip, the working tool may be any working tool such as, for example, a spacer, a balloon actuator, a bone tamp, etc.

1. An apparatus, comprising:
a first clamp having a first end and a second end, the second end of the first clamp configured to engage a first spinous process;
a second clamp having a first end and a second end, the second end of the second clamp configured to engage a second spinous process spaced apart from the first spinous process; and
a connector coupled to the first end of the first clamp and the first end of the second clamp.

2. The apparatus of claim 1, wherein the connector is reconfigurable between a first position and a second position, the first clamp being a first distance from the second clamp when the connector is in the first position and being a second distance from the second clamp when the connector is in the second position.

3. The apparatus of claim 1, wherein the second end of the first clamp includes a first jaw and a second jaw opposite the first jaw, the first jaw and the second jaw configured to be movable between a first configuration and a second configuration, the first jaw and second jaw being closer together in the second configuration than in the first configuration.

4. The apparatus of claim 1, the first clamp including a first jaw and a second jaw at the second end, the apparatus further comprising a tension member coupled to the first connector, a distance between the first jaw and the second jaw being changed based on a movement of the tension member.

5. The apparatus of claim 1, wherein the first clamp includes a first arm and a second arm, the first arm being pivotably coupled to the second arm, the first arm being separable from the second arm at the second end.

6. The apparatus of claim 1, further comprising:
a swing arm pivotably coupled to the connector, the swing arm having a predefined range of motion.

7. The apparatus of claim 1, further comprising:
a swing arm pivotably coupled to the connector, the swing arm having a first end and a second end, the second end of the swing arm configured to receive a working tool.

8. The apparatus of claim 1, further comprising:
a swing arm pivotably coupled to the connector, the swing arm having a first end and a second end, the second end of the swing arm including a pointed projection, the pointed projection configured to define a pathway in tissue.

9. The apparatus of claim 1, further comprising:
a swing arm having a first end and a second end, the first end of the swing arm pivotably coupled to the connector, the swing arm defining an opening along its length;
a working tool configured to engage the second end of the swing arm;
a guide wire having a first end and a second end, the second end of the guide wire coupled to the working tool, the opening of the swing arm configured to receive the guide wire; and
a retainer coupled to the second end of the guide wire, the retainer configured to be removably coupled to the swing arm.

10. The apparatus of claim 1, further comprising:
a swing arm having a substantially arcuate shape, the swing arm having a range of motion including a position between the first spinous process and the second spinous process when the swing arm is pivotably coupled to the connector.

11. An apparatus, comprising:
an arcuate body portion having a first end and a second end, a side wall extending between the first end and the second end, the side wall defining a passageway between the first end and the second end;
a working tool configured to engage the second end;
a guide wire having a first end and second end, the first end of the guide wire coupled to the working tool, the guide wire configured to be received in the opening; and
a retainer coupled to the second end of the guide wire, the retainer configured to be removably coupled to the body portion adjacent the first end of the apparatus.

12. The apparatus of claim 11, wherein the side wall defines a slot along its length between the first end and the second end.

13. The apparatus of claim 11, further comprising a working tool removably coupled to the second end, the working tool being one from a plurality of interchangeable working tools.

14. A method, comprising:
coupling a first clamp to a first spinous process;
coupling a second clamp to a second spinous process spaced apart from the first spinous process; and
changing a distance between the first spinous process and the second spinous process based on a movement of at least one of the first clamp and the second clamp.

15. The method of claim 13, further comprising:
inserting a portion of a swing arm between the first spinous process and the second spinous process after the coupling the first clamp, the coupling the second clamp and the changing the distance.

16. The method of claim 13, further comprising:
inserting a swing arm at a position between the first spinous process and the second spinous process after the coupling the first clamp, the coupling the second clamp and the changing the distance; and
inserting a spacer at the position.

17. The method of claim 13, further comprising:
defining a path that includes a position between the first spinous process and the second spinous process; and
inserting along the path a spacer configured to be located at the position.

18. A method, comprising:
moving an arcuate body between adjacent spinous processes of a vertebra to define an opening within tissue disposed between the adjacent spinous processes, the arcuate body having a second end portion configured to receive a working tool;
removing the arcuate body from the opening;
coupling a spacer to the arcuate body; and
inserting the spacer in the opening using the arcuate body.
